(19) United States
(12) Patent Application Publication
(10) Pub. No.: US 2013/0032145 A1
(43) Pub. Date: Feb. 7, 2013

(54) DRY POWDER INHALER

(71) Applicants: Dan Adler, Karkur (IL); Amnon Kadron Kritzman, Karkur (IL); Amir Genosar, Boulder, CO (US); Amnon Slutski, D.N. Beit Shaan (IL)

(72) Inventors: Dan Adler, Karkur (IL); Amnon Kadron Kritzman, Karkur (IL); Amir Genosar, Boulder, CO (US); Amnon Slutski, D.N. Beit Shaan (IL)

(21) Appl. No.: 12/989,649

(22) PCT Filed: Apr. 28, 2009
(86) PCT No.: PCT/IL09/00451

§ 371 Date: May 4, 2011

(30) Foreign Application Priority Data
May 1, 2008 (IL) 191.190

(51) Int. Cl. A61M 15/00

(52) U.S. Cl. 128/203.15

(57) ABSTRACT

A dry-powder inhaler device comprising an air inlet (40), an air outlet (4), an assembly comprising a mesh-based package (10) containing a dry-powder drug (16), the assembly being positioned to undergo agitation, the device having an airflow therethrough from the air inlet to the outlet, which airflow at least indirectly causes the agitation and wherein the agitation of the assembly in conjunction with the airflow causes the particles of the dry-powder drug to exit from the mesh-based package and become entrained in the airflow.

### FIELD OF THE INVENTION

This invention relates to a dry powder inhaler based on a mesh-package for delivering dry-powder drugs. Specifically, the present invention describes an inhaler employing an active mesh-based package for the dry-powder drug, and methods for agitating said package in order to release the dry-powder from the package.

### BACKGROUND OF THE INVENTION

Numerous drugs, medications and other substances are inhaled into the lungs for rapid absorption in the blood stream. Inhaled drugs fall into two main categories: (1) liquids, including suspensions; and (2) powders. The present invention relates to the latter category.

Dry-powder inhalers need to deliver a particle size that is predominantly below 5 microns, and preferably between 1 micron and 3.3 microns, for maximum effectiveness. Such small particles are, however, thermodynamically unstable due to their high surface area to volume ratio, which provides significant excess surface free energy and encourages particles to agglomerate. In an inhaler, agglomeration of small particles is a problem which results in the active particles leaving the inhaler as large agglomerates.

The role of meshes in deagglomeration of particles is known in the prior art. For example, U.S. Pat. No. 6,871,647 describes an inhaler in which a mesh is incorporated into the drug powder compartments and where the drug powder is entrained by the air flow through said mesh. Similarly, U.S. Pat. No. 5,388,572 describes a mesh disc impregnated with drug powder doses, where the air flow is produced by a piston which produces an air pressure blast. In further prior art publications, Wang, et al. in the Journal of Aerosol Medicine, Vol. 19, No. 2, 2006, pp. 168-174, describe the effect of a sieving mesh on an inhaler's performance. According to this inhaler design, when users draw a breath from the mouthpiece, they cause dry powder medicine to swirl, creating a cyclone. Larger clumps of powder are pounded into tiny particles when they collide with one another and against a fine wire mesh with a 38 μm pore size.

However, although these approaches incorporate a mesh in the encapsulation of the powder dose, the mesh is only used as a passive sieve against which powder particles are driven by air pressure, rather than playing a more active role. Accordingly, the disaggregation achieved is very limited.

In view of these drawbacks and limitations of the prior art, what is needed is a dry powder inhaler device in which effective disaggregation of the dry powder particles is achieved.

Therefore, it is an object of the invention to provide a simple inhaler device which achieves good disaggregation of the dry powder released.

It is a further object of the invention to provide a dry-powder inhaler which synchronizes the drug release with the inhalation action of the patient.

It is a still further object of the invention to spread the delivery of the dry-powder over a defined duration of the breath.

It is a still further object of the invention to prevent dry-powder particles being retained in the capsule/device; thereby achieving a high emitted dose.

It is a still further object of the invention to power the inhalation action purely by use of the patient's breath power.

It is a further object of the invention to provide a convenient and portable housing for said inhaler.

It is a still further object of the invention to exploit aeroelastic phenomena resulting from the airflow through the inhaler device to produce agitation.

It is further the object of the invention to provide a device that enables the transporting of the drug separate from the device such that the patient can load said drug into the device.

It is further the object of the invention to provide a device that is indifferent to accidental air-blow into the device.

These and other objects of the present invention are achieved in the preferred embodiments disclosed below by providing a dry-powder inhaler based on a special type of drug powder packaging.

### SUMMARY OF THE INVENTION

The inhaler device of the present invention provides an improved and simplified mechanism for dry powder drug inhalation, which ensures the synchronization of fine-particle release during inhalation. This device employs a mesh-based package to store the dry-powder drug, where said mesh package plays an active role in causing the disaggregation of the dry powder particles. The functioning of the device involves the agitation of said package in order to release the dry-powder through the holes in the mesh. Without wishing to be bound by theory, the operation of said agitation can be likened to the beating of a carpet where powder particles resident on said carpet are mobilized away from the carpet; in this case thereupon to be swept up in a rapid airflow in the direction of the air outlet of the inhaler device. Unlike “powder fluidization” type approaches such as that described in U.S. Pat. No. 6,889,690, in which the powder particles are oscillated but left substantially in place (relying on the passing air stream to remove them), the approach of the current invention is to force the particles to migrate through the mesh package enclosing them by vibrations and beating which cause significant inertia forces to act on the particles. At the same time, air flow through the package may optionally be employed to enhance this effect and entrain the particles into the air, inhaled by the patient. Thus this form of agitation produces a beating type of effect, and this beating and/or agitating of the mesh is referred to herein as an “active mesh” approach. A number of different mechanisms or agitating means for producing this type of agitation are described in detail below. In a preferred embodiment, said agitating means are powered purely by the inhalation action of the patient.

Thus according to the present invention, there is now provided a dry-powder inhaler device comprising an air inlet, an air outlet, an assembly comprising a mesh-based package containing a dry-powder drug, said assembly being positioned to undergo agitation, said device having an airflow therethrough from said air inlet to said outlet, which airflow at least indirectly causes said agitation and wherein the agitation of said assembly in conjunction with said airflow causes the particles of said dry-powder drug to exit from said mesh-based package and become entrained in said airflow. In preferred embodiments of the present invention, said agitation is initiated by an airflow which causes vibration due to an aeroelastic effect. In this case the airflow directly initiates the vibration and/or agitation.

Preferably, said agitation is enhanced by a Venturi effect.

In some preferred embodiments of the present invention said agitation involves vibrating said assembly such that said assembly repeatedly strikes a rigid surface, thereby causing said dry-powder drug particles to be entrained into the airflow.

In preferred embodiments of the present said assembly is associated with a spring.

Preferably said device further comprises agitating means wherein said agitating means is a breath-driven rotor comprising an axis and rotor blades, wherein said rotor blades repeatedly impact said spring which, in turn causes a beating of the attached mesh-based package, thereby causing said dry-powder drug particles to exit said package and be entrained into the airflow. In this case the airflow drives said rotor and thus the agitation of the assembly is caused indirectly by said airflow.

In some preferred embodiments of the present invention said spring is attached to said assembly and said agitation means repeatedly strikes said spring. In other preferred embodiments of the present said agitation means repeatedly strikes the mesh-based package of said assembly.

Preferably said mesh-based package is formed from mesh types selected from the group consisting of woven meshes and perforated meshes.

In preferred embodiments of the present invention said mesh-based package is shaped according to designs selected from sandwich shapes and cylindrical shapes.

Preferably said package is filled via a vibrating funnel.

The mesh-based package may be any type of package with appropriate sized holes. Examples include packages fabricated from netting, woven-style meshes where the holes exist by virtue of the weaving structure (i.e. are located between the threads), perforated materials and laser-perforated materials, etc. Said materials may be fabricated from plastic or metals, with the use of materials such as aluminum or aluminized foil currently used for forming dry powder blisters being advantageous from a regulatory perspective. All packages constructed from such materials are herein termed “mesh-based packages” or “package”.

In the present invention the holes in said mesh-based packages are of a size sufficiently large to enable the exit of the particles as the package is agitated, but small enough to significantly resist their exit under gravity alone. For example, for a 3 micron diameter particle, the preferred hole size is between 6 microns and 150 microns, more preferably between 10 microns and 80 microns and most preferably between 20 microns and 50 microns. The thickness of the mesh is typically the same as or a small multiple of the hole size for optimal functionality. For example, a typical mesh-based package with holes of 40 microns in size may have a thickness of between 40 microns and 240 microns. Again, without being limited by theory, having the thickness of the package material be larger than the hole size provides the advantages of (a) enhancing the ability of the mesh-based package to retain the dry powder particle until use; and (b) enabling the delay between the onset of agitation and the start of particle release to be determined so that the optimal drug-delivery profile can be designed. At its simplest, the particles require a greater number of agitations or beatings in order to find their way to the outside of the package when the package material is thicker; there being a tunneling effect which takes more time. Said tunneling effect also serves to improve the disaggregation that takes place as the particles are agitated out of the package.

Advantageously, the fact that the dry-powder particles remain within the package until it is agitated or beaten obviates the requirement for a capsule-based package as is typically used in dry-powder inhalers. In capsule-based inhalers the powder is stored in a hermetically sealed capsule, said capsule needing to be opened in a step prior to use of the inhaler. This opening step is disadvantageous, not only because of the requirement for an extra action, but also because some of the powder is typically is trapped within the capsule, thereby reducing the emitted dose at the outset. When using a mesh-based package, the inhaler device can be ready for use without a capsule insertion and/or opening step, and the hermetic or modified atmosphere sealing can be provided around the entire device. As shown in one of the embodiments below, this can greatly simplify the overall operation of a single-dose inhaler device. Additionally, the use of a mesh-based package greatly reduces the amount of powder (if any) that is left inside said package once it has been agitated and flushed by the inhaled air.

The agitation or beating of the mesh-based package can be provided in a number of ways within the inhaler device of the present invention. In particular, the package can constitute part or all of an assembly such as a leaf, reed or spring which is agitated, either directly or indirectly, by the airflow through the device, said airflow constituting the agitation means using an aeroelastic effect. In its simplest form, the assembly incorporates a spring, reed or leaf which, due to an aeroelastic effect, is vibrated by the airflow through the inhaler device. In some embodiments, these vibrations may in turn cause said assembly to repeatedly impact against a rigid surface. The vibration and/or impaction causes the powder particles to emerge from the mesh-based package and become entrained in the airflow through the device. This effect may be further enhanced by at least part of the airflow penetrating and flushing the package and thus assisting in entraining the powder particles. Examples of device embodiments in which this simple design is used include those based on a whistle or harmonica type embodiments, and those where a Venturi principle is utilized. More complex embodiments involve the indirect application of a repeated impaction action to the assembly. In such embodiments, the patient's intake breath causes breath-driven motion of a moving assembly where, as said moving assembly repeatedly agitates the assembly, the mesh-based package portion of said assembly releases powder particles as described above. An example of said moving assembly is a rotor or turbine, which is an agitation means driven by the patient's intake breath.

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

### BRIEF DESCRIPTION OF THE FIGURES

FIGS. 1a and 1b provide isometric exploded and assembled views respectively of a flat embodiment of the mesh-based package of the present invention;

FIGS. 1c and 1d provide isometric exploded and assembled views respectively of a cylindrical embodiment of the mesh-based package of the present invention;

FIG. 2a shows a cross-sectional view of the incorporation of said package into a reed-shape design of the assembly of the present invention;

FIG. 2b shows a cross-sectional view of the incorporation of said flat mesh package into a spring-shape design of the assembly of the present invention;

FIG. 2c shows a cross-sectional view of the incorporation of said cylindrical mesh package into a spring-shape design of the assembly of the present invention;

FIGS. 3a, 3b and 3c respectively provide a series of cross-sectional views of the mesh-based package as said package is agitated or beaten, showing the migration of the powder particles out of the package;

FIG. 4 provides a cross-sectional view of a Venturi-based embodiment of the inhaler of the present invention incorporating a reed-shaped assembly;

FIG. 5a provides a cross-sectional view of an embodiment of the inhaler of the present invention incorporating a reed-shaped assembly;

FIG. 5b provides a planar view of an embodiment of the inhaler of the present invention incorporating a reed-shaped assembly;

FIG. 6a provides a planar interior view of a rotor-based embodiment of the inhaler of the present invention incorporating a curved, spring-shaped assembly prior to the rotor blade striking said spring-shaped assembly;

FIG. 6b provides a planar interior view of a rotor-based embodiment of the inhaler of the present invention incorporating a curved, spring-shaped assembly directly subsequent to the rotor blade striking said spring-shaped assembly, showing the powder released by this action;

FIG. 6c provides a planar interior view of said rotor-based embodiment; further comprising a removable cover over its air outlet;

FIGS. 7a and 7b respectively show isometric exploded and assembled views of the embodiment provided in FIG. 6c;

FIG. 8 provides a planar view of a packaging system for the reed-based embodiment; and

FIG. 9 shows a planar view of a filling method for a mesh-based package according to the present invention.

### DETAILED DESCRIPTION OF THE FIGURES

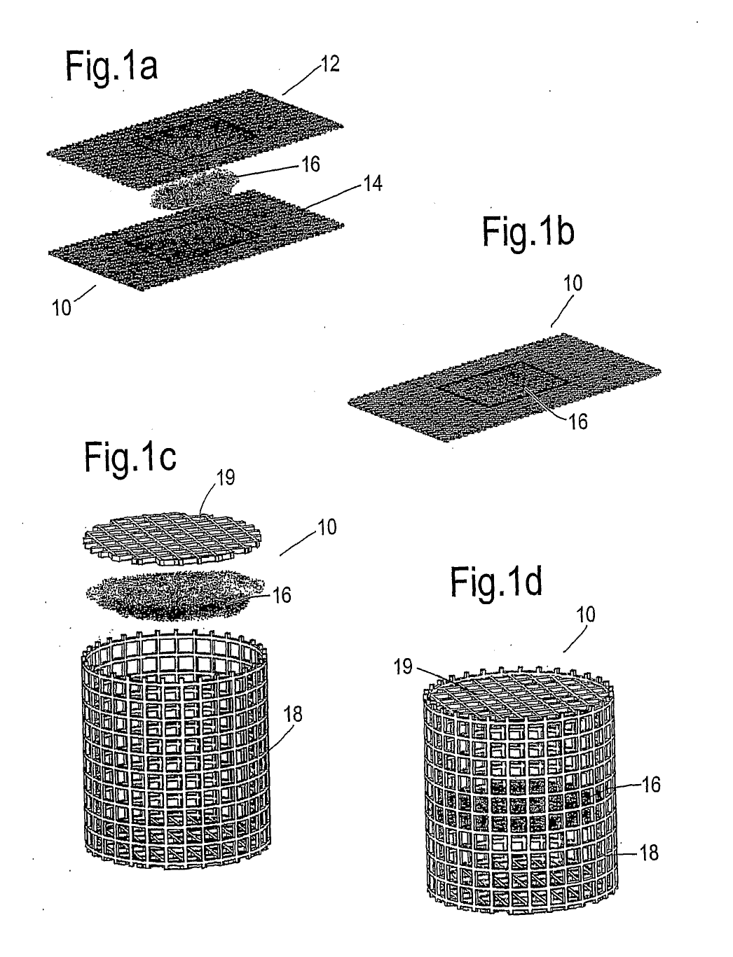

FIGS. 1a and 1b provide isometric exploded and assembled views respectively of flat and cylindrical embodiments of the mesh-based package of the present invention. As described above, the mesh-based package employed by the present invention can contain a free flowing drug powder constrained within a mesh. The exploded diagram in FIG. 1a illustrates a preferred embodiment of a sandwich-type design of a suitable mesh-based package 10 for use according to this approach. In this sandwich type design, the drug powder 16 is entrapped between a first mesh layer 12 and a second mesh layer 14. Suitable materials for the meshes include both polymeric materials and metal meshes such as MicroMesh® electroformed meshes from Precision Eforming LLC, Cortland, N.Y., USA, and mesh or TPS sieve material from Tecan Ltd., Weymouth, Dorset, UK. From a regulatory perspective, dry-powder inhaler blisters are currently fabricated from aluminum foil, so there is an advantage to forming such meshes from aluminum or similar metals.

Referring now to FIG. 1b, the assembled sandwich type mesh-based package 10 is shown, with the drug powder 16 entrapped within it. The package may be held together by a number of means, including but not limited to adhesion, crimping of the metal, folding, etc. In a further preferred embodiment, the package may be formed by applying an external frame (not shown) around at least part of the package, to hold it together. For example, a frame may be fabricated from a plastic material such as PVC, PET or PE together with an adhesive layer; such that applying pressure and/or heat to the outer frame of the sandwich serves to seal the sandwich as a whole. Such a frame would require leaving an open window for the drug particles to emerge from when the package is agitated. Thus, it is apparent to one skilled in the art, that the mesh-based package 10 of the present invention does not need to be solely formed using mesh, but may also comprise additional materials as part of its fabrication. For example, in a further preferred embodiment said additional materials may comprise a solid tube-shaped component to direct the airflow; potentially implementing a cone to concentrate said airflow.

Likewise, it will be apparent to one skilled in the art that the sandwich shape shown above is not the only shape that could be employed for the embodiment of the mesh-based package. Other potential shapes include cubes, prisms, cylinders, etc. For example, FIG. 1c illustrates a cylindrical preferred embodiment for the mesh package 10, comprising an open-topped cylinder 18 fabricated at least partially from mesh material, into or onto which a disk or cover 19 is placed in order to close the package 10 after filling said package 10 with the dry-powder 16 drug. The assembled version of this preferred embodiment is shown in FIG. 1d. Advantageously, by presenting a deeper cavity for the drug 16 to be filled into, this embodiment makes the filling process simpler, as is further discussed below.

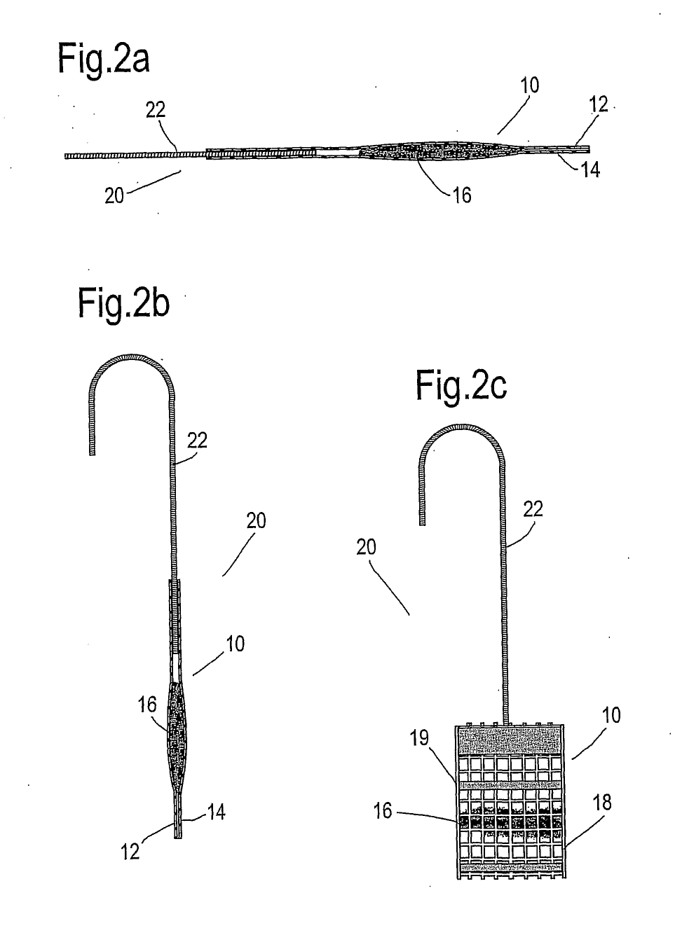

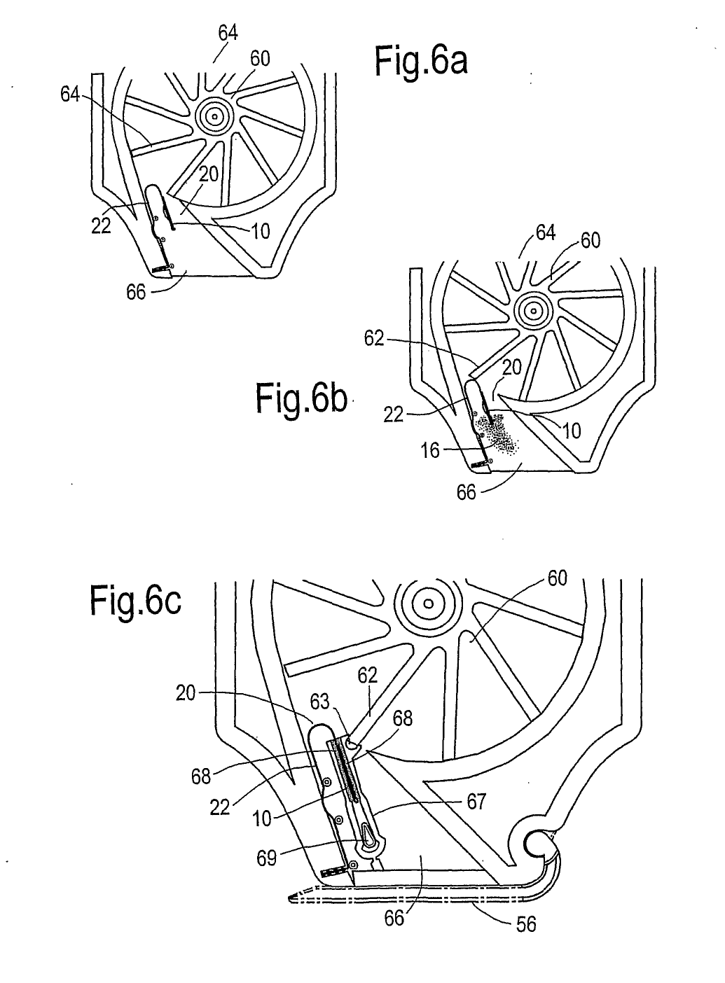

The assembly of the present invention may be entirely formed by the mesh-based package or, in a further preferred embodiment; the mesh-based package may constitute just one component of said assembly. In the former case, there is a reliance on the mesh itself to provide whatever structural support or springiness is required for the functioning of the assembly as a whole. In the latter case, the structural, springy or vibrational element can be supplied by an additional component comprising the assembly. Referring now to FIGS. 2a, 2b and 2c, some exemplary embodiments of the assembly of the present invention are shown. FIG. 2a illustrates a simple preferred embodiment of a reed-shaped assembly 20 comprising a spring 22 attached to the mesh-based package 10; such that agitation of the assembly 20 as a whole will cause the powder 16 to exit the mesh. Said agitation can be caused by a further moving member striking any part of the assembly, or vibration of the assembly due to an aeroelastic effect. Referring now to FIG. 2b, the spring 22 has a curved shape and is connected to a sandwich shaped mesh-based package 10. In the further preferred embodiment shown in FIG. 2c, the curved spring 22 is connected to a cylinder shaped mesh-based package 10. The spring material may be either metal or plastic. Advantageously, where a long curved spring is used, as shown in FIGS. 2b and 2c, the point of agitation may be far removed from the point of powder release; and practical embodiments employing this advantage are shown in FIG. 6 below.

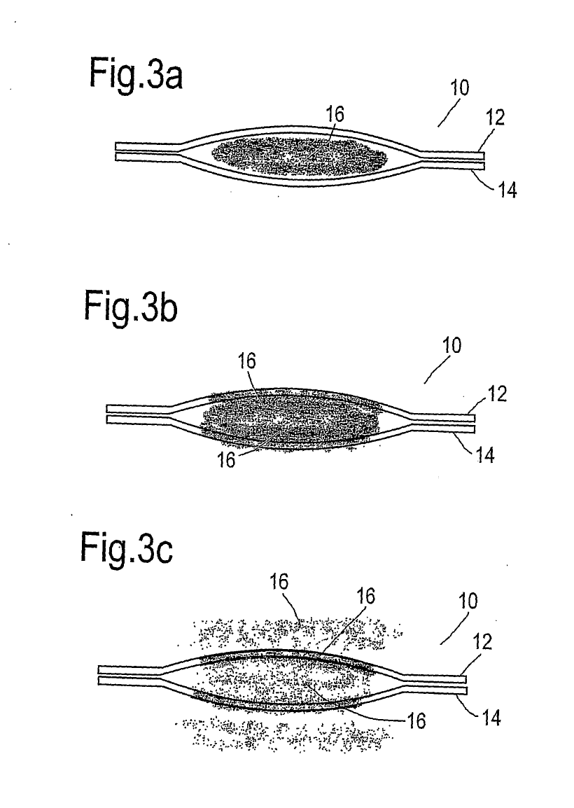

Referring now to FIGS. 3a, 3b and 3c, a series of cut-away cross-sectional views of the mesh-based package 10 of the present invention are shown in order to illustrate the effect of the agitation of said package. The initial, rest state of the package is shown in FIG. 3a, where all the drug powder 16 is located in the space between the first mesh layer 12 and the second mesh layer 14. As the package is agitated, the powder particles start to disperse into the mesh layers (12, 14) as shown in FIG. 3b. Subsequently, on further agitation, the powder particles 16 start to emerge from the mesh layers, as shown in FIG. 3c, and entrained into the airflow. Without wishing to be bound by theory, the rate of release of the drug can be controlled by altering parameters of the meshes used and of the agitation applied (for example the intensity and/or orientation of said agitation). Thus a mesh with smaller holes and/or smaller open area will constrain the powder to being entrained more slowly and a mesh with larger holes and/or a larger open area will enable the drug release to be speeded up. Another parameter that can be altered is the depth of the holes. To the extent that a perforated mesh is used, the depth of the holes is determined by the thickness of the perforated material, and the time taken (or number of agitations required) for particles to emerge from a deeper hole is longer. This effect can be used to provide a delayed onset for the start of the particle release, a clinical effect that has been shown to be of some importance, for example in the article, “Dose Delivery Late in the Breath Can Increase Dry Powder Aerosol Penetration into the Lungs”, Bondesson, et. al., Journal of Aerosol Medicine; Mar. 1, 2005, 18(1): pp 23-33. Whereas such an effect of timing the powder delivery to occur late in the breath is currently available in sophisticated electronically-controlled inhalers, the implementation of such an effect by merely changing a parameter of the mesh material is a significant advance in the art.

It will be noted that the embodiment shown in FIG. 3a has a capsule shape and thus is well adapted to be encapsulated within a secondary sealing layer, as is known in the dry-powder packaging art. Dry powder drug particles are typically hygroscopic and thus it is important to enclose any and all of the types of mesh-based packages described above within some form of blister arrangement. Such blisters typically comprise an aluminum foil. Numerous mechanical arrangements may be made to remove said aluminum outer foil directly before the inhaler of the present invention is used by the patient, for example by pulling out said foil via the air inlet or outlet. In the case where a particulate filter is present in the air outlet, then the air inlet is preferred. In a multi-dose embodiment or any other embodiment where the (next) mesh-based package is inserted manually, the arrangement may be such that the insertion action causes the foil to be stripped away. The inhaler of the present invention may also or alternatively be sealed externally by such a foil, preferably with a controlled atmosphere inside the device.

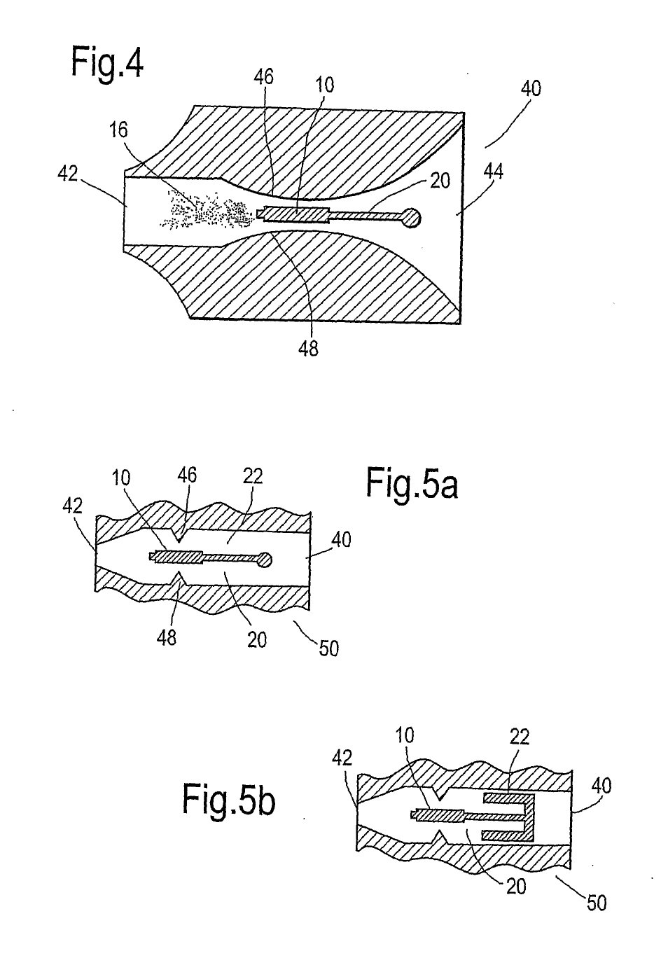

Referring now to FIG. 4, a preferred, Venturi-based embodiment of the inhaler of the present invention incorporating a reed-shaped assembly is shown. The Venturi effect is an example of Bernoulli's principle, in the case of incompressible flow through a flow passage with a constriction in it. The fluid velocity must increase through the constriction to satisfy the equation of continuity, while its pressure must decrease due to conservation of energy: the gain in kinetic energy being supplied by a drop in pressure or a pressure gradient force. This effect is used in the modern day barrel of the clarinet, which uses a reverse taper to speed the air down the tube, enabling better tone, response and intonation. In the inhaler device of the present invention, the increase of velocity in the airflow 44 between the air inlet 40 and the air outlet 42 causes the reed-shaped assembly 20 (described in FIG. 2a above) to be agitated by aeroelastic effect at an enhanced speed. In a preferred embodiment, said agitation of the reed 20 causes the mesh-shaped package at its end to repeatedly strike the rigid surfaces 46 and 48, thereby causing the powder particles 16 to be entrained into the airflow 44. Advantageously, the high speed of the airflow serves to reduce the probability that these particles will be retained on said surfaces 46 and 48.

Referring now to FIGS. 5a and 5b, a further preferred embodiment of the inhaler device of the present invention is shown. In this preferred embodiment, the device 50 is a capsule-shaped one, formed from plastic or preferably aluminum foil. FIG. 5a provides a cross-sectional view of said device 50, showing an air inlet 40 and air outlet 42, with a reed or leaf-shaped assembly located within; such that the agitation of said assembly due to the aeroelastic effect of the airflow through the capsule 50 causes the assembly to vibrate and release particles. In a preferred embodiment, as said assembly 20 vibrates, its mesh-based package 10 component repeatedly strikes the rigid surfaces 46 and 48, causing the powder within said package 10 to be released and entrained into the airflow. Advantageously, a capsule-shaped inhaler device such as that shown may be very inexpensively fabricated, and is very suitable for incorporation into blister packaging such as that typically used for capsules or pills. Referring now to FIG. 5b, a preferred method of anchoring the spring portion of the assembly 20 is shown; where the side arms of the spring 22 contact the sides of the capsule wall.

In the above embodiments of the inhaler device of the present invention, the airflow through the inhaler causes the assembly to become agitated, thereby causing powder particles to be released from the mesh-based package. However, there are numerous further embodiments in which the assembly is impacted by a further moving member; all such embodiments falling within the scope of the present invention. For example, referring now to FIG. 6a, a planar view of .a breath-driven, rotor-based embodiment of the inhaler of the present invention is shown; said preferred embodiment comprising a rotor 60 mounted on as axis (not shown) and a curved assembly 20 positioned such that its mesh-based package 10 is located close to the air outlet 66. The rotor blade tips 62 are arranged such that, as the rotor 60 rotates, said tip 62 is positioned so that it will make contact with the curved portion of the spring 22 each time that the tip 62 reaches said spring 20. Due to the flexibility of this spring 22, the blade tip 62 knocks the spring 22 sideways enabling said tip 62 to continue in its rotation. In this manner, the assembly 20 receives a beating each time a rotor tip 62 makes contact with it. Referring now to FIG. 6b, the beating action of the rotor tip 62 against the assembly 22 is shown, said action causing drug particles 16 to be beaten out of the mesh-based package 10, and thus this beating action (together with in any vibration induced) is the primary entraining action of the inhaler. As described in connection with the embodiments described above, the thus entrained drug powder particles 16 are swept up into the airflow through the air outlet path 66 and inhaled. Furthermore, the proximity of the mesh-based package 10 to the outlet 66 of the device and the high speed of the airflow ensure that said drug powder particles 16 have very limited opportunity to become aggregated before they are inhaled. As will be obvious to one skilled in the art, the orientation of the mesh-based package 10 to the airflow through the device also plays a part in the entraining action. To the extent that said package 10 is somewhat perpendicular to the air outlet path 66, this may increase the extent to which the airflow passes through said package 10, which may further enhance the entraining effect achieved by a flushing effect.

Referring now to FIG. 6c, a preferred embodiment of a removable closure 65 for opening the inhaler of the present invention until use is illustrated. In said preferred embodiment said closure 65 comprises a lever 61 for opening said closure and a clip 67 connected to said closure 65. Said clip 67 further comprises a socket 63 for a rotor blade tip 62, and two elastic or spongy pads 68, one either side of the mesh-based package 10; such that when the closure 65 is in its closed position (shown), said socket 63 prevents the rotor turning and said pads 68 prevent the mesh-based package 10 from experiencing any kind of unwanted agitation prior to use. Such unwanted agitation may result from dropping or knocking the device. On opening of this closure 65, the clip 67 is opened by the cam 69, such that the pads 68 release the mesh-based package 10. At the same time, the opening of this closure 65 frees the rotor tip 62 from its socket 63, such that the rotor 60 is free to turn, and the assembly 20 is free to be agitated; and thus the inhaler is ready for use.

Thus, it will be realized that in a preferred embodiment of the present invention there is provided a closure 65 across said air outlet 66, said closure comprising pads 68 serving to prevent the mesh-based package from undergoing agitation as a result of an externally applied impact.

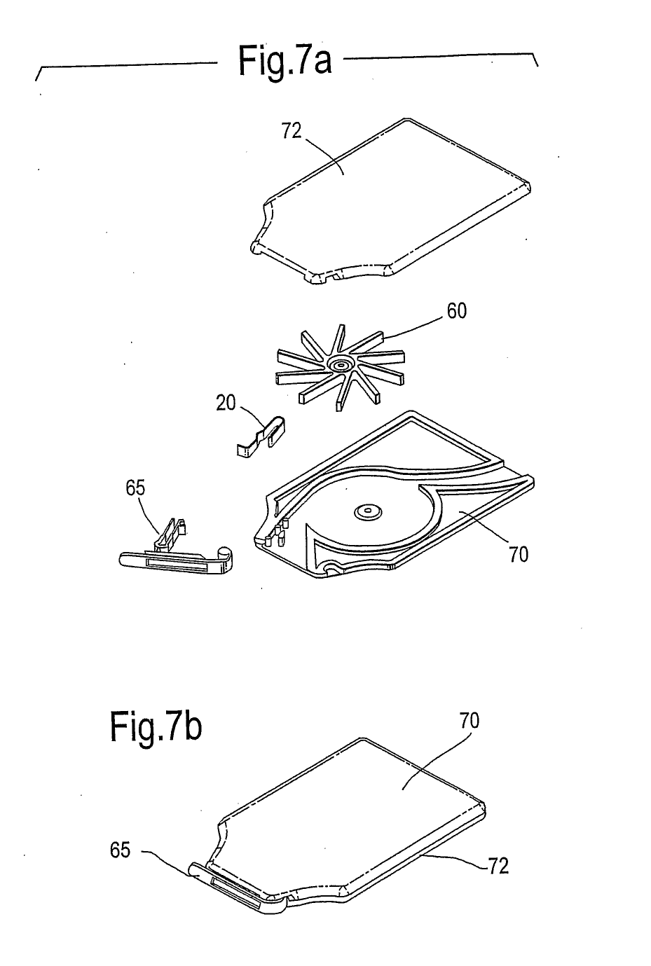

FIGS. 7a and 7b provide exploded and assembled isometric views of the rotor-based embodiment of the inhaler device respectively. As seen in FIG. 7a, the chassis 70 of the device is adapted to receive the rotor 60, the assembly 20 and the closure 65; where the addition of a cover 72 completes the device. The assembled version of this device is shown in FIG. 7b. Note that said device can be fabricated to be small and slim, preferably with a form factor similar to that of a credit-card, which is a convenient and popular shape. As will be obvious to one skilled in the art, such a shape is very easy to package in a hermetically sealed fashion, and thus this embodiment is simple to seal using just a controlled atmosphere and an external seal. In a further preferred embodiment, the device may be sealed by a tape such as a metalized tape covering the air outlet and air inlet(s). In a preferred embodiment said tape will have a tab for easy removing before using the device. As long as the embodiment is a single-dose one, these methods of sealing are sufficient. Note however, that if a multi-dose version of the inhaler device is required, then each dose (i.e. each mesh-based package) will need to be separately enclosed in a sealed capsule as described above.

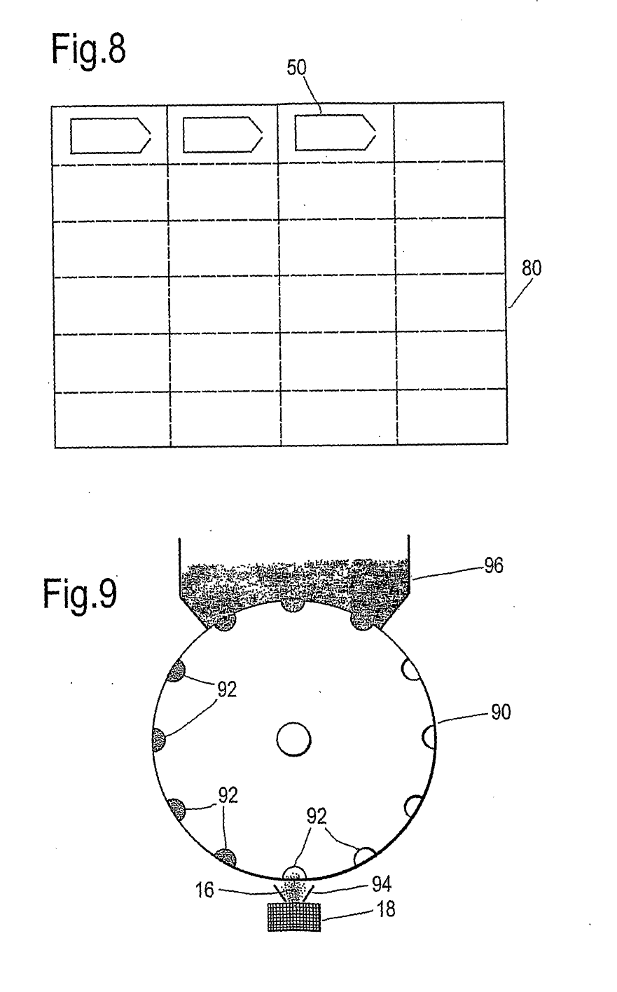

A number of different packaging configurations of the inhaler of the present invention can be offered. For example, given the credit-card shape described above in FIGS. 6c and 7b, a product design according to this embodiment can be easily stacked and boxed. If however, the capsule configuration described in FIGS. 5a and 5b is employed, products designed according to this embodiment are more naturally packaged using a standard blister-type packaging approach. Advantageously, said blister packaging is well known in the pharmaceutical packaging field, and is capable of providing the high-barrier environment required in order to preserve the dry-powder drugs. Referring now to FIG. 8, a preferred embodiment of such blister packaging 80 is shown, where the capsule shaped inhaler devices 50 of the present invention are stored, one per blister. Each such inhaler device is a single-dose, disposable inhaler, which is popped out of its blister directly before use. In this way, the device itself does not need to be a sealed unit, as the sealing is provided by the blister packaging.

A major issue with dry-powder inhalers is the question of rapid filling in an automated fashion. A standard method known in the art is to use a drum-filling machine such as that shown in FIG. 9, whereby a drug powder hopper 96 serves to fill cavities 92 in a rotating drum 90; where said drum cavities 92 then deposit their powder contents into a blister cavity beneath the drum. Such blister cavities are typically about 7 mm across and thus a puck of powder material, typically measuring 1.5-2.5 mm across can be emptied into such a blister cavity without requiring great tolerances. However, in the case of the inhaler of the present invention, the mesh-based package into which the powder dose needs to be filled can be of much smaller dimensions, for example 1-2 mm across. In order to ensure that the puck is loaded into the mesh-based package, it is advantageous to use the cylinder type package 18 described in FIGS. 1c and 1d and a vibrating funnel 94 to funnel the powder into the waiting open-topped cylinder 18. By using high-frequency vibrations, for example above 1000 Hz, the powder is channeled into the package without being significantly retained on the walls of the funnel. In a preferred embodiment, the upper diameter of the funnel 94 will be approximately 7 mm, while the lower one will be approximately 1-2 mm, where the lower end of said funnel 94 is slightly below the upper rim of the cylinder 18. The filling action is then completed by having the next station on the automated filling line close the cylinder 18 from above as shown in FIG. 1d. In order to fill a sandwich-type mesh-based package, said sandwich is partially sealed in advance, with a non-sealed seam being left (typically on one side). The filling can then proceed by holding said package sideways, with the open seam facing upwards, and filling into this seam using the funnel method described above. Thus, in both cases, the small mesh-based packages of the current invention can be filled while only slightly modifying current dry-powder filling lines.

As will be obvious to one skilled in the art, the basic operating principle of the inhaler of the present invention whereby the agitation of the assembly serves to release dry-powder drugs from the mesh-based package portion of said assembly can, in addition, be enhanced by the additional application of various additional means known in the art. For example, various aerodynamic techniques to optimize the airflow through the inhaler may be used, the shape of the inhaler may be optimized so as to minimize the extent to which some of the dose is trapped on the container walls, and electronic vibrations (for example using a piezoelectric transducer) may be used to minimize agglomeration by adding a “powder fluidization” effect. In other words, a combination of an aeroelastic (or mechanical) agitation effect may be combined with a piezoelectric vibration effect, although it should be noted that such a product would not then be powered solely by the patient's breath. Alternatively or additionally, the aeroelastic effect may be used to produce a plurality of vibration frequencies, each one playing a different role in entraining the drug powder particles.

As will be apparent to those skilled in the art, by using the same basic operating principle but with different mechanical arrangements, a number of further embodiments of the inhaler of the present invention can be produced. Examples of such further preferred embodiments include multi-dose inhalers where a new mesh-based package is moved into position between each dose, and multiple-use inhalers where a fresh dose is inserted manually into the device before each use. Suitable shapes for the inhaler device of the present invention include those similar to credit-cards, whistles or candy bars.

Suitable medicaments for use in the invention include any drug or drugs which may be administered by inhalation and which is either a solid or may be incorporated in a solid carrier. Suitable drugs include those for the treatment of respiratory disorders, e.g., bronchodilators, corticosteroids and drugs for the prophylaxis of asthma. Other drugs such as anorectics, anti-depressants, anti-hypertensive agents, anti-neoplastic agents, anti-cholinergic agents, dopaminergic agents, narcotic analgesics, beta-adrenergic blocking agents, prostoglandins, sympathomimetics, tranquilizers, steroids, vitamins and sex hormones may be employed. Exemplary drugs include: Salbutamol, Terbutaline, Rimiterol, Fentanyl, Fenoterol, Pirbuterol, Reproterol, Adrenaline, Isoprenaline, Ociprenaline, Ipratropium, Beclomethasone, Betamethasone, Budesonide, Disodium Cromoglycate, Nedocromil Sodium, Ergotamine, Salmeterol, Fluticasone, Formoterol, Insulin, Atropine, Prednisolone, Benzphetamine, Chlorphentermine, Amitriptyline, Imipramine, Cloridine, Actinomycin C, Bromocriptine, Buprenorphine, Propranolol, Lacicortone, Hydrocortisone, Fluocinolone, Triamcinclone, Dinoprost, Xylometazoline, Diazepam, Lorazepam, Folic acid, Nicotinamide, Clenbuterol, Bitolterol, Ethinyloestradiol and Levenorgestrel. Drugs may be formulated as a free base, one or more pharmaceutically acceptable salts or a mixture thereof

It will be evident to those skilled in the art that the invention is not limited to the details of the foregoing illustrative embodiments and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A dry-powder inhaler device comprising an air inlet, an air outlet, an assembly comprising a mesh-based package containing a dry-powder drug, said assembly being positioned to undergo agitation, said device having an airflow therethrough from said air inlet to said outlet, which airflow at least indirectly causes said agitation and wherein the agitation of said assembly in conjunction with said airflow causes the particles of said dry-powder drug to exit from said mesh-based package and become entrained in said airflow.

2. The inhaler device of claim 1 wherein said agitation is initiated by an airflow which causes vibration due to an aeroelastic effect.

3. The inhaler device of claim 2 where said agitation is enhanced by a Venturi effect.

4. The inhaler device of claim 1 where said agitation involves vibrating said assembly such that said assembly repeatedly strikes a rigid surface, thereby causing said dry-powder drug particles to be entrained into the airflow.

5. The inhaler device of claim 1 wherein said assembly is associated with a spring.

6. The inhaler device of claim 5 further comprising agitating means, wherein said agitating means is a breath-driven rotor comprising an axis and rotor blades, wherein said rotor blades repeatedly impact said spring which, in turn causes a beating of the attached mesh-based package, thereby causing said dry-powder drug particles to exit said package and be entrained into the airflow.

7. The inhaler device of claim 6 wherein said spring is attached to said assembly and said agitation means repeatedly strikes said spring.

8. The inhaler device of claim 6 where said agitation means repeatedly strikes the mesh-based package of said assembly.

9. The inhaler device of claim 1 where said mesh-based package is formed from mesh types selected from the group consisting of woven meshes and perforated meshes.

10. The inhaler device of claim 1 where said mesh-based package is shaped according to designs selected from sandwich shapes and cylindrical shapes.

11. The mesh-based package of claim 1 where said package is filled via a vibrating funnel.

12. The inhaler device of claim 1 where a multiplicity of said inhalers is packaged in a blister package.

13. The inhaler device of claim 1 where said inhaler is shaped like a credit-card

14. The inhaler device of claim 1 where said inhaler is shaped like a whistle.

15. The inhaler device of claim 1 where said inhaler is a disposable single-dose inhaler.

16. The inhaler device of claim 15 where said inhaler is sealed externally and contains a controlled atmosphere.

17. The inhaler device of claim 1 where said inhaler is a re-useable inhaler that is loaded externally.

18. The inhaler device of claim 1 where said inhaler is a multi-dose inhaler where a new mesh-based package is moved into position between each dose.

19. The inhaler device of claim 6, further comprising a closure across said air outlet, said closure serving to prevent the rotor turning.

20. The inhaler device of claim 6, further comprising a closure across said air outlet, said closure comprising pads serving to prevent the mesh-based package from undergoing agitation as a result of an externally applied impact.

21. The inhaler device of claim 2 wherein said aeroelastic effect operates on said assembly.
